(19) United States
(12) Patent Application Publication
(10) Pub. No.: US 2010/0198111 A1
(43) Pub. Date: Aug. 5, 2010

(54) METHOD FOR INFLUENCING THE PRONATION BEHAVIOUR OF A SHOE

(75) Inventors: Thomas Milani, Markkleeberg (DE); Thorsten Sterzing, Chemnitz (DE); Stephan Odenwald, Chemnitz (DE); Ralph Doerfler, Chemnitz (DE)

Correspondence Address: LUCAS & MERCANTI, LLP, 475 PARK AVENUE SOUTH, 15TH FLOOR, NEW YORK, NY, 10016, US

(73) Assignee: PUMA AKTIENGESELLSCHAFT RUDOLF DASSLER SPORT, Herzogenaurach (DE)

(21) Appl. No.: 12/601,344

(22) PCT Filed: Dec. 8, 2008
(86) PCT No.: PCT/EP2008/010374

§ 371 Date: Apr. 6, 2010

(30) Foreign Application Priority Data
Dec. 29, 2007 (DE) 10 2007 063 160.1

(51) Int. Cl. A61B 5/103

(52) U.S. Cl. 600/592

(57) ABSTRACT

The invention relates to a method for influencing the pronation behaviour of a shoe (1), especially of a sports shoe, in which a) at least on parameter (φ, dφ/dt) is measured (2) which is relevant for the behaviour of pronation, b) the measured parameter (φ, dφ/dt) is led to a control unit (3), c) the control unit (3) outputs an actuation signal (S) which is influencing the behaviour of pronation to an actuator (4) and d) the actuator (4) changes a property of the shoe (1) which property is relevant for the behaviour of pronation. To better influence the behaviour of pronation the invention is characterized in that the processing of data of a parameter (φ, dφ/dt) measured according to step a) takes place from a reference point of time (t_{K}), wherein the reference point of time is determined from the touch down of the shoe (1) on the ground.

The invention relates to a method for influencing the pronation behaviour of a shoe, especially of a sports shoe, in which
a) at least on parameter is measured which is relevant for the behaviour of pronation,
b) the measured parameter is led to a control unit,
c) the control unit outputs an actuation signal which is influencing the behaviour of pronation to an actuator and
d) the actuator changes a property of the shoe which property is relevant for the behaviour of pronation.

In the state of the art systems are known which are integrated in shoes, especially in sport shoes, by which a property of the shoe can be influenced actively to influence the pronation. Those systems are operated with the method of the generic typ. For example, the springing behaviour and damping behaviour respectively of the shoe can be influenced. For doing so, certain movement conditions of the shoe are measured and the springing behaviour and damping behaviour respectively of the shoe are adjusted selectively by means of influenceable elements. A solution of such a kind is disclosed e.g. in U.S. Pat. No. 5,813,142.

In the mentioned document a sensor system for the evaluation of the pressure in a chamber is provided which chamber is integrated in the shoe sole and can be charged with a fluid. A control device controls the supply of a medium into the fluid chamber in dependence from the measured pressure.

In general, with such a system the effect of controlling the pronation of the shoe can be influenced—what is very desirable—when the shoe touches down on the ground.

The pronation is a rotation of the foot around the axis of the lower ankle joint by which the outer edge of the foot is lifted and the inner edge of the foot is lowered. The pronation is also called an inward-rotation or inward-edging.

The normal pronation of the foot is a natural damping mechanism and a natural movement inwardly during the touch down of the foot. However, the edge of the foot flexes strongly inwardly in the case of the so-called over-pronation and thus stresses the ligaments, the tendons and the joints. This over-pronation can have different reasons, e.g. a defective position of the foot, overweight or strong exhaustion. Also, over-pronation occasionally occurs in the case of running beginners, because the supporting apparatus of the foot is not yet trained sufficiently. Then, a strong wear can be seen at the shoes in the medial region.

The mechanism which is antipodal to the pronation (also called supination) occurs seldom during running. In the case of supination the load is directed in the opposite direction. This can be seen at running shoes by a higher wear in the lateral region (i.e. at the outer side).

Consequently, it is aimed with modern sports shoes to carry out an active influence of the pronation beyond the pronation supports which are integrated in the sole and which are known as such what is possible with a system as described in the mentioned document.

The known systems are switched on when demanded and work continuously to as long as it is necessary, i.e. during the whole period of the use of the shoe.

Consequently, during the whole switch-on period data are measured, processed in a processor and an actuator is operated. Thus, the system is also active when the foot does not at all touch the ground during running.

Thereby, it is a problem to measure or to determine the relevant data which are in fact relevant for the influence of the pronation, by which the pronation can be influenced effectively. Those data cannot be isolated in a simple manner from the parameters which otherwise determine the running dynamics.

Thus, it is the object of the invention to further develop a method of the kind mentioned at the beginning so that is becomes possible to determine relevant data for the measurement of the pronation in an improved and simpler manner. So, the determination of the data and the influence of the pronation should be led back to a better reference to thereby effect an improved controlling of the pronation. Thereby, this should be possible in an especially easy manner.

The solution of this object is characterized by the invention in that the processing of data of a parameter measured according to above step a) takes place from a reference point of time, wherein the reference point of time is determined from the touch down of the shoe on the ground.

Thereby, the reference point of time can be the contact point of time of the shoe on the ground by itself or can be dependent from the same.

Preferably, the touch down of the shoe on the ground can be detected by a sensor which is integrated into the shoe and which outputs a signal to the control unit which is characteristic for the touch down.

A pressure or force sensor can be used as the sensor, which measures the pressure or the force respectively which is exerted by the foot of the wearer of the shoe on the sole of the shoe.

Alternatively, an acceleration sensor can be used as the sensor, which can measure the acceleration or deceleration respectively of the shoe during touch down of the shoe on the ground.

According to a further alternative the sensor can also be a range sensor, which measures the deformation of the sole during the touch down of the shoe on the ground, by which the beginning ground contact can be detected.

The sensor is arranged preferably in the heel region of the shoe. It can be arranged in the sole between the bottom side of the foot of the wearer and the ground contact area of the sole. Especially, the sensor can be arranged between an outer sole and a midsole of the shoe.

The processing of data of a parameter measured according to above step a) can be terminated when the lift off of the shoe from the ground is detected. Consequently, the utilization of the measured data takes place substantially during the period, in which the shoe has contact with the ground.

Alternatively, it is also possible that the processing of data of a parameter measured according to above step a) is terminated when a predetermined time from the reference point of time is elapsed. For example, it can be provided here that the utilization of the data in the meaning explained above takes place from the contact point of time of the shoe on the ground for 250 ms and subsequently the processing of data is terminated.

After the end of the processing of data of a parameter measured according to above step a) the actuator can be driven into a reference or zero position or generally into an appropriate or calculated position respectively.

Although the processing of measured values does not occur permanently according to the invention it can be provided however that the measurement of parameters which are relevant for the behaviour of pronation according to above step a) takes place permanently, especially also during phases in which no contact exists between the shoe and the ground. Thus, in this case only in certain time intervals measured values are utilized which enter permanently.

According to a further development of the invention it can be provided in this case, that the control unit outputs controlling signals to the actuator incorporating measured data of parameters which are already measured within a defined period prior the touch down of the shoe on the ground. Thus, if measured data are collected permanently, it can for example be provided that measured data are incorporated for the controlling of the pronation which have been already measured in a period of time of 10 ms prior the touch down of the shoe on the ground.

A parameter which is relevant for the behaviour of pronation can be the angle or the angular speed of the shoe around a predetermined axis.

The measurement of the angle or the angular speed of the shoe around the axis can be affected by means of a gyroscopic sensor which is arranged at or in the shoe.

Preferably, the projection of the axis on the ground area includes an angle between 0° and 45° with the longitudinal axis of the shoe, especially an angle between 0° and 10°. Meanwhile, the axis can, seen in the direction of the longitudinal axis, include an angle between 0° and 70° with the ground area, especially an angle between 0° and 10°.

The relevant property for the behaviour of pronation of the shoe can be the thickness of the sole between the bottom side of the foot of the wearer and the ground contact area of the sole in a defined region of the width of the shoe transverse to the longitudinal axis of the shoe. However, it is also possible that the relevant property for the behaviour of pronation of the shoe is the spring rigidity of the sole in a defined region of the width of the shoe transverse to the longitudinal axis of the shoe. Furthermore, it is possible that the relevant property for the behaviour of pronation of the shoe is the angular position between the upper and the bottom side of the sole.

It can be achieved by the proposed embodiment of a shoe and by the mentioned method for influencing of the pronation respectively that those measured data are utilized in an improved manner which are specifically relevant for the influence of the pronation. The basis is the point of time in which ground contact between the shoe and the ground is given, wherein the parameter values are then utilized for the control of the pronation for the time of ground contact or for a defined period of time which are measured permanently.

Furthermore, an energy-saving operation exists when the control of the pronation takes place only in certain phases of a stride cycle.

Thus, the point of time of the contact of the shoe on the ground serves as a trigger for the influencing of the pronation in the explained meaning. For example, the signal of contacting can then activate the pronation control system, wherein however the measurement of parameters occurs permanently.

In the drawing an embodiment of the invention is shown.

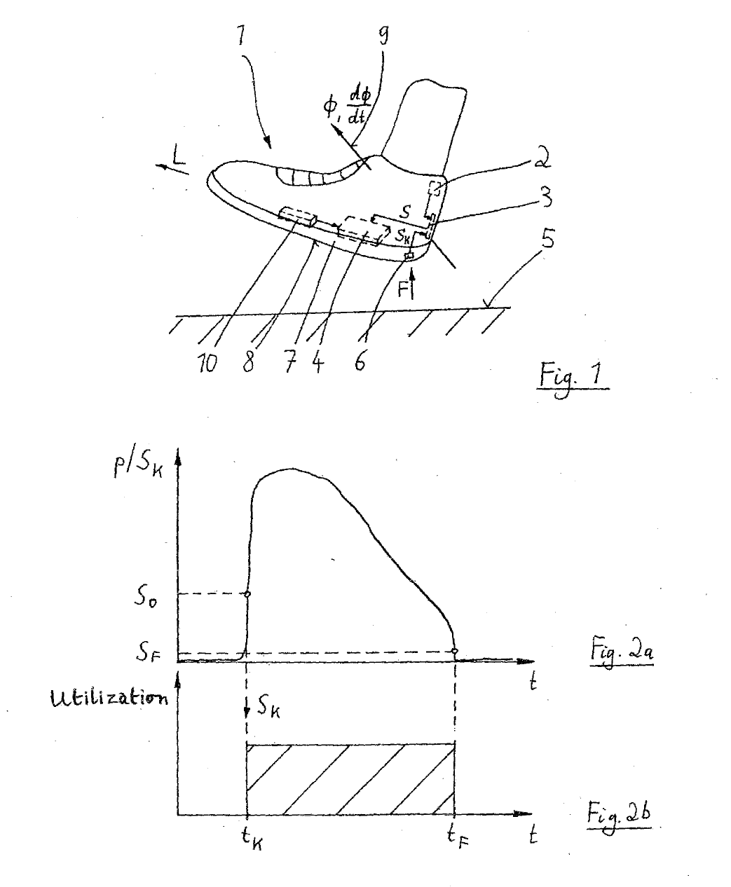

FIG. 1 shows schematically the leg of a runner with a shoe, shortly before the touch down of the shoe on the ground,

FIG. 2a shows the run of a measured value of a sensor for the detection of the touch down of the shoe on the ground over time and

FIG. 2b shows the associated run of the activity, i.e. of the operation of a system for influencing of the pronation over time.

In FIG. 1 the bottom part of a leg of a runner can be seen who wears a shoe 1. During a stride and during a stride cycle respectively the shoe 1 is airborne for a certain time. Then it touches down on the ground 5 and rolls off on the same, before it lifts off again. The shoe 1 has a sole 7 with a ground contact area 8. Depicted is the situation shortly prior the touch down of the shoe 1 on the ground 5, i.e. the shoe 1 is here still in the flight phase.

The shoe 1 is equipped with a system 2, 3, 4, 10 for influencing of the pronation of the foot of the wearer of the shoe 1. This system comprises at first a sensor 2, which is able to detect a parameter which is relevant for the pronation. Presently, the angular speed dφ/dt of the shoe as the derivation of an angle φ is applied as relevant parameter. The angular speed dφ/dt is measured around an axis 9, which is inclined under an angle to the longitudinal axis L of the shoe 1 as well as to the horizontal. It is proven to use a gyroscopic sensor as the sensor 2 which is able to deliver a signal which is proportional to the angular speed.

This signal (measured value) is forwarded from the sensor 2 to a control unit 3 with microprocessor, in which a controlling and feed-back-controlling algorithm respectively is stored. In dependence on the determined measured value the control unit 3 initiates the output of an actuation signal S to an actuator 4 based on the stored algorithm, i.e. to an actuator which is able to change a parameter of the shoe 1, which is relevant for the pronation, in such a manner that the pronation can be influenced selectively.

Thereby, especially an actuator 4 is suggested which can change the effective thickness of the sole 7 between the contact area of the foot on the sole and the ground contact area 8 in a lateral region of the sole 7. Consequently, the foot tilts during the touch down on the ground and during the roll off of the foot on the ground respectively more or less around the longitudinal axis L of the shoe 1. In general, also an actuator can be employed which influences the spring or damping properties of the shoe as it is known as such in the state of the art.

Thereby, the actuator 4 receives energy from a battery 10 to carry out the respective actuation movements.

It is essential, that the processing of data of the parameter φ and dφ/dt respectively takes place from a reference point of time t_{K} (see FIG. 2b), wherein this reference point of time is determined from the touch down of the shoe 1 on the ground.

For the determination of this point of time t_{K} a sensor 6 is arranged in the shoe 1 and namely in its sole 7, preferably between an outer sole and a midsole. This sensor can be a pressure sensor and a force sensor respectively which is able to measure the force F (or a pressure p) which acts between the foot of the wearer and the ground 5. During the flight phase no contact exists between the shoe and the ground 5 so that the force, which is measured by the sensor 6, is substantially Zero. Meanwhile, touches the shoe down on the ground 5 after the termination of the flight phase the sensor 6 registers a value.

The measured value of the sensor 6 it transmitted to the control unit 3 as a contact signal S_{K}. As soon as a signal value is detected, which lies above a threshold limit value S₀, the data, which are measured by the sensor 2, are utilized to actively influence the pronation by means of the system 2, 3, 4, 10.

This is depicted in FIG. 2a and FIG. 2b. In FIG. 2a the run of the measured value of the sensor 6 is shown as a function over time. Thus, at the ordinate the (qualitative) magnitude of the sensor signal is plotted. As soon as the measured force and the measured pressure respectively exceeds the threshold limit value S₀ the utilization of the measured data takes place. The measurement of the data by itself can take place permanently.

This is indicated in FIG. 2b. At the point of time t_{K} the excess of the threshold limit value S₀ is given, so that the utilization of the measured data starts (shaded area of the utilization of the measured data in FIG. 2b). When the sensor value drops again to a smaller, predetermined value S_{F} (start of the flight phase) this is also registered by the control unit 3, which thereupon terminates the utilization of measured data.

Meanwhile, the actuation movements of the pronation controlling system take place mostly during the flight phase of the shoe. During this time no measured data at all are utilized by the control unit according to the invention.

The sensor 2 for the evaluation of a parameter which is relevant for the pronation is designed here as a gyroscopic measurement system, which is able to measure an angular speed dφ/dt around the axis 9. The axis 9 is arranged in a certain position in the shoe which is especially important for the determination of the pronation of the shoe and the foot of the wearer respectively. Its ground projection is arranged under an angle to the longitudinal axis L of the shoe 1, which is approximately 0° till 10°. If the axis 9 is regarded in the direction of the longitudinal axis L it includes an angle to the horizontal, which also is in the region between 0° and 10°. The sensor 2 is arranged in the lateral region of the shoe 1, namely in the lower region of the shoe upper.

The sensor 6 is arranged preferably—as already mentioned—between an outer sole and a midsole of the shoe 1, namely directly at the posterior edge of the shoe (i.e. in the rear edge region).

Not depicted arc further sensors 2 when indicated, wherein a sensor is suggested which is located for example in the heel region posterior of the metatarsal bridge which can be an acceleration sensor and which can measure an acceleration in the direction of the horizontal and transverse to the longitudinal axis L. Thus, the acceleration sensor then detects a horizontal acceleration in medio-lateral direction.

In general, the proposed method for influencing of the extent of movement and of the velocity of movement is suitable especially for the shoe heel region, i.e. for influencing of the extent and the velocity of the pronation.

As explained, with the sensor 6 the initial ground contact of the shoe 1 can be detected. A deflection of the sensor signal (at the point of time t_{K} in FIG. 2) shows that the ground contact of the shoe and especially of the heel starts. Correspondingly, a drop of the pressure of the measured value (at the time t_{F} in FIG. 2) signals a lift off of the shoe from the ground 5.

To obtain the rotational speed of the heel movement the gyroscopic measurement system 2 is used for the measurement of the angular speed dφ/dt. The detected maximum corresponds to the maximum pronation speed.

By the integration of the signal of the angular speed dφ/dt which is measured by the gyroscopic sensor 2 the extent of the angle of the pronation can be determined. Correspondingly, by the integration of the signal of the angular speed over the time the run of the (pronation) angle can be determined. The difference between the minimum and the maximum delivers the extent of the pronation.

It is also possible to carry out the utilization of the measured data for a predetermined period after the point of time t_{K}, e.g. for a period of 250 ins. Also it is possible—as far as the collection of measured data takes place permanently—to access data for the utilization of measured data which already lie prior the point of time t_{K}, e.g. within a period of 10 ms prior t_{K}.

Furthermore, it is also possible to gather the measured values which are collected in that way for a number of strides and then incorporate them as averaged values for the controlling of the pronation.

While the proposed method is preferably used in a system which is integrated in the shoe and takes care there for an active influencing of the pronation behaviour it is in general also possible to use the proposed process for stationary and mobile measurements, e.g. for the analyses of the running performance of a runner (e.g. on a threadmill).

### REFERENCE NUMERALS

1 Shoe
2 Sensor (gyroscopic sensor)
3 Control unit
4 Actuator
5 Ground
6 Sensor
7 Sole
8 Ground contact area
9 Axis
10 Energy source (battery)
S Actuation signal
S₀ Threshold limit value
S_{K} Signal for the touch down of the shoe on the ground
F Force
L Longitudinal axis of the shoe
φ, dφ/dt Parameter
φ Angle
dφ/dt Angular speed
t_{K} Reference point of time

1. Method for influencing the pronation behaviour of a shoe, in which
at least one parameter is measured which is relevant for the behaviour of pronation,
b) the measured parameter is led to a control unit,
c) the control unit outputs an actuation signal which is influencing the behaviour of pronation to an actuator and
d) the actuator changes a property of the shoe which property is relevant for the behaviour of pronation,
wherein
the processing of data of the parameter measured according to step a) takes place from a reference point of time, wherein the reference point of time is determined from the touch down of the shoe on the ground.

2. Method according to claim 1, wherein the touch down of the shoe on the ground is detected by a sensor which is integrated into the shoe and which outputs a signal to the control unit which is characteristic for the touch down.

3. Method according to claim 2, wherein a pressure or force sensor is used as the sensor, which measures the pressure or the force respectively which is exerted by the foot of the wearer of the shoe on the sole of the shoe.

4. Method according to claim 2, wherein an acceleration sensor is used as the sensor, which can measure the acceleration or deceleration respectively of the shoe during touch down of the shoe on the ground.

5. Method according to one of claim 2, wherein the sensor is arranged in the heel region of the shoe.

6. Method according to claim 2, wherein the sensor is arranged in the sole between the bottom side of the foot of the wearer and the ground contact area of the sole.

7. Method according to claim 6, wherein the sensor is arranged between an outer sole and a midsole of the shoe.

8. Method according to claim 1, wherein the processing of data of the parameter measured according to step a) of claim 1 is terminated when the lift off of the shoe from the ground is detected.

9. Method according to claim 1, wherein the processing of data of the parameter measured according to step a) of claim 1 is terminated when a predetermined time from the reference point of time is elapsed.

10. Method according to claim 8, wherein after the end of the processing of data of the parameter measured according to step a) of claim 1 the actuator is driven into a defined position or into a reference or zero position.

11. Method according to claim 1, wherein the measurement of the parameters which are relevant for the behaviour of pronation according to step a) of claim 1 occurs permanently, especially also during the phases in which no contact exists between the shoe and the ground.

12. Method according to claim 11, wherein the control unit outputs controlling signals to the actuator incorporating measured data of the parameters which are already measured within a defined period prior the touch down of the shoe on the ground.

13. Method according to claim 1, wherein the parameter which is relevant for the behaviour of pronation is the angle or the angular speed of the shoe around a predetermined axis.

14. Method according to claim 13, wherein the measurement of the angle or the angular speed of the shoe around the axis is affected by means of a gyroscopic sensor which is arranged at or in the shoe.

15. Method according to claim 13, wherein the projection of the axis on the ground area includes an angle between 0° and 45° with the longitudinal axis of the shoe.

16. Method according to claim 13, wherein the axis, seen in the direction of the longitudinal axis, includes an angle between 0° and 70° with the ground area.

17. Method according to claim 1, wherein the relevant property for the behaviour of pronation of the shoe is the thickness of the sole between the bottom side of the foot of the wearer and the ground contact area of the sole in a defined region of the width of the shoe transverse to the longitudinal axis of the shoe.

18. Method according to claim 1, wherein the relevant property for the behaviour of pronation of the shoe is the spring rigidity of the sole in a defined region of the width of the shoe transverse to the longitudinal axis of the shoe.
